(19) United States
(12) Patent Application Publication
(10) Pub. No.: US 2021/0269213 A1
(43) Pub. Date: Sep. 2, 2021

(54) SELF-SEALING TISSUE STORAGE CONTAINER

(71) Applicant: Instant Systems, Inc., Norfolk, VA (US)

(72) Inventors: Kimber L. BURLEY, Fulton, MD (US); Tara C. RAMSEY, Norfolk, VA (US)

(73) Assignee: Instant Systems, Inc., Norfolk, VA (US)

(21) Appl. No.: 17/258,695

(22) PCT Filed: Jul. 8, 2019
(86) PCT No.: PCT/US2019/040892

§ 371 Date: Jan. 7, 2021

Related U.S. Application Data
(60) Provisional application No. 62/695,669, filed on Jul. 9, 2018.

(51) Int. Cl. B65D 75/30; B65D 81/32; B65D 75/58; A01N 1/02; A61F 2/00

(52) U.S. Cl. CPC B65D 75/30; B65D 81/3266; A61F 2/0095; B65D 75/5805; A01N 1/0263; B65D 75/5855

(57) ABSTRACT

An apparatus includes a container that is robust for storing tissue and does not require secondary containment. The container includes a self-sealing portion that allows the container to be sealed at a variety of locations (e.g., a surgical suite or the like) without specialized equipment. The self-sealing portion can include any suitable mechanism.

### CROSS-REFERENCE TO RELATED APPLICATIONS

This application claims benefit of priority to U.S. Provisional Application No. 62/695,669, entitled “Self-Sealing Tissue Storage Container,” filed Jul. 9, 2018, which is incorporated herein by reference in its entirety.

### BACKGROUND

The embodiments described herein relate to containers for storing and transporting tissue and other biological material. More particularly, the embodiments described herein relate to devices and methods including self-sealing, flexible containers that can withstand the conditions needed for storing and transporting tissue.

Known tissue implants and/or grafts are used in a variety of procedures to repair or replace damaged tissue. Such procedures can include implanting bone or gum tissue to address dental or periodontal issues, bone grafting to repair fractures, and tendon grafting to repair damaged ligaments and/or tendons (e.g., repair of a torn anterior cruciate ligament), to name just a few. In many instances, the tissue implant is not taken from the patient's body (i.e., is not an autograft), but rather is from another source, such as from a human cadaver (i.e., an allograft) or an animal (i.e., a xenograft). Tissue is also used in a variety of research or training applications. Thus, known methods for preparing tissue implants can include multiple handling operations. Such operations can include recovering donated tissue, packaging the donated tissue for shipment to a processing center, preparation and processing of the donated tissue, and storage of the donated tissue for later shipment to a recipient.

During certain recovery operations, the tissue may be frozen, refrigerated, cryopreserved, and/or stored at ambient temperatures. The tissue may also be transported using various refrigerants, such as, for example, wet ice, dry ice, LN2 filled or LN2 vapor phase storage transport boxes. In other situations, the tissue may not be refrigerated or cooled during transportation. As such, effective tissue recovery operations require specialized containers (or container systems) that can be used in a variety of different situations. Such container systems keep the tissue therein free from contact with (or contamination by) outside sources (e.g., refrigerants, other tissues, etc.) during storage, handling, and transportation.

Unfortunately, known tissue handling procedures can result in loss of tissue due to insufficient container systems. For example, some known containers are not robust enough to prevent breaches (e.g., such known containers are susceptible to rips, tears, inadvertent opening, etc.). To limit the likelihood of breach when using flexible containers, some known container systems include one or more secondary containers to protect the inner container and contents. Such secondary containers, however, are often rigid, and therefore can be difficult to manipulate and pack, and can also inefficiently use the available space in the shippers/freezers (which can increase shipping cost). Moreover, known sealing systems and procedures that are used at the point of recovery are often insufficient to limit breach. Specifically, although known sealing methods in a centralized setting can include sophisticated heat sealers, these devices can be bulky and expensive and are therefore not suitable for use at a point of recovery. Accordingly, some known methods for sealing a tissue recovery container include simply folding (or twisting) a portion of the container upon itself (one or more times) and then securing the container with a clasp or zip tie. Such methods are not repeatable, are highly dependent on the technique used by the operator (e.g., how tightly the container is twisted or folded), etc.

Thus, a need exists for improved containers and methods for storing and transporting tissue and other biological material.

### SUMMARY

Containers and methods for storing tissue and other biological materials are described herein. In some embodiments, an apparatus includes a container that is robust for storing tissue and does not require secondary containment. The container includes a self-sealing portion that allows the container to be sealed at a variety of locations (e.g., a surgical suite or the like) without specialized equipment. The self-sealing portion can include any suitable mechanism that provides a repeatable seal that limits penetration of microbes therethrough.

In some embodiments, the self-sealing portion can include a series of sealing mechanisms, such as plastic zippers, that collectively produce a tortuous path to reduce the penetration of microbes. In some embodiments, the self-sealing portion can include a tamper evident feature that provides an indication of whether the container has been reopened after initially being sealed.

In some embodiments, the container can include gussets or other structural members such that the container is self-standing.

In some embodiments, an apparatus includes a first layer of flexible material and a second layer of flexible material. The second layer is coupled to the first layer to form a container defining a storage volume, and the storage volume is bounded by a first end portion of the container, a second end portion of the container, a first side edge of the container, and a second side edge of the container. The first and second layers of flexible material define an opening along a segment extending along the first end portion and the first side edge, and the first and second layers together include a self-sealing portion along the segment extending along the first end portion and the first side edge. In some embodiments, the apparatus includes a port, and the port is configured to allow fluid communication between an outside volume and the storage volume defined between the first and second layers of material. In some embodiments, the segment extending along the first end portion and the first side edge includes a curved segment section. In some embodiments, the segment extending along the first end portion and the first side edge includes a linear segment section and a curved segment section. In some embodiments, the segment extending along the first end portion and the first edge includes a first linear segment section, a second linear segment section, and a curved segment section. The curved segment section is disposed between the first linear segment section and the second linear segment section.

In some embodiments, the second end portion includes a tear notch on the first side edge and a perforated tear strip extending between the notch and the second side edge. In some embodiments, the self-sealing portion includes a set of sealing mechanisms. The set of sealing mechanisms includes any one of a zipper or an adhesive. In some embodiments, at least one of the set of sealing mechanisms is configured to be a single-use sealing mechanism, and the self-sealing portion includes an indicator that identifies when the set of sealing mechanisms are in a sealed configuration. In some embodiments, at least one of the set of sealing mechanisms is configured to be a single-use sealing mechanism, and the self-sealing portion includes an indicator that identifies when the set of sealing mechanisms have been opened after an initial instance of being fully sealed.

In some embodiments, an apparatus comprises a first layer of flexible material, a second layer of flexible material, and a self-sealing portion. The second layer is coupled to the first layer to form a container defining a storage volume. The storage volume is bounded by a first end portion of the container a second end portion of the container, and a set of side edges of the container. The first layer and the second layer define an opening at the first end portion of the container. The self-sealing portion includes set of sealing mechanisms, each of the set of sealing mechanisms being configured to close and seal the opening at the first end portion. In some embodiments, the first layer and the second layer are configured to be coupled at a central portion of the container to separate the storage volume into a first partition and a second partition. The central portion is located between the first end portion and the second end portion.

In some embodiments, the apparatus further comprises a second self-sealing portion at the central portion of the container. The second self-sealing portion includes a first seal member and a second seal member. The first seal member is configured to matingly engage the second seal member to fluidically isolate the first partition from the second partition. The first seal member includes a base portion, a neck portion and a head portion. The neck portion is disposed between the base portion and the head portion. In some embodiments, the first seal member is attached to an exterior surface of the first layer, opposite of the storage volume. In some embodiments, the first seal member is attached to the exterior surface of the first layer via the head portion. In some embodiments, the second seal member includes a base portion, a first shoulder, a second shoulder, and a groove defined between the first shoulder and the second shoulder. In some embodiments, the second seal member is attached to an exterior surface of the second layer, opposite the storage volume. The second seal member is attached to the exterior surface of the second layer via one of the first shoulder or the second shoulder. In some embodiments, the groove of the second seal member is configured to receive and retain the head portion of the first seal member, the first layer at the central portion of the container, and the second layer at the central portion of the container.

In some embodiments, the apparatus further comprises a third self-sealing portion at the second end portion, the third self-sealing portion including a first seal member and a second seal member. In some embodiments, the self-sealing portion comprises a set of complimentary hook and grooves and a set of complimentary U-shaped ribs. The set of complimentary hook and grooves is a first set of complimentary hook and grooves, and the self-sealing portion comprises a second set of complimentary hook and grooves. In some embodiments, the apparatus includes a port, and the port is configured to allow fluid communication between an outside volume and the storage volume defined between the first and second layers.

In some embodiments, at least one of the set of sealing mechanisms is configured to be a single-use sealing mechanism, and the self-sealing portion includes an indicator that identifies when the set of sealing mechanisms are in a sealed configuration. In some embodiments, at least one of the set of sealing mechanisms is configured to be a single-use sealing mechanism, and the self-sealing portion includes an indicator that identifies when the set of sealing mechanisms have been opened after an initial instance of being fully sealed.

In some embodiments, an apparatus comprises a first layer of flexible material, a second layer of flexible material, a third layer of flexible material, and a self-sealing portion. The second layer of flexible material is coupled to the first layer to form a container bounded by a first end portion of the container, a second end portion of the container and a set of side edges of the container. The first layer and the second layer define an opening at the first end portion. The third layer of flexible material is attached to and configured to selectively decouple from at least one of the first layer or the second layer, and the third layer of flexible material is disposed between the first layer and the second layer to define a first storage volume of the container between the first layer and the third layer. A second storage volume of the container is defined between the second layer and the third layer. In some embodiments, the self-sealing portion has a set of sealing mechanisms to close and seal the opening at the first end portion. In some embodiments, at least one of the set of sealing mechanisms is configured to be a single-use sealing mechanism, and the self-sealing portion includes an indicator that identifies when the set of sealing mechanisms are in a sealed configuration. In some embodiments, the apparatus further comprises a port configured to allow fluid communication between an outside volume and one or more of a first storage volume or a second storage volume defined between the first and second layers. In some embodiments, the apparatus further comprises a port configured to allow fluid communication between an outside volume and the storage volume defined between the first and second layers of material. In some embodiments, the apparatus further comprises a first flap portion attached to the first layer at the first end portion and a second flap portion attached to the third layer at the first end portion, the first and second flap portions extending beyond the opening at the first end portion. In some embodiments, the first flap portion and the second flap portion form a continuous sleeve or funnel. In some embodiments, the continuous sleeve or funnel encircles at least a portion of the opening at the first end portion.

In some embodiments, the self-sealing portion includes a first sealing member secured to the first layer. The self-sealing portion includes a second sealing member secured to the second layer. The first sealing member and the second sealing member are configured to interlock together to seal the opening at the first end portion. In some embodiments, the first sealing member includes a first hook and groove channel, and the second sealing member includes a first corresponding hook and groove channel configured to interlock with the hook and groove channel of the first sealing member. The first sealing member includes a second hook and groove channel, and the second sealing member includes a second corresponding hook and groove channel configured to interlock with the second hook and groove channel of the first sealing member.

In some embodiments, the second layer is secured to a first side of the second sealing member, and the third layer is secured to a second side of the second sealing member, opposite of the first side of the second sealing member. In some embodiments, the second sealing member includes a groove and the third layer is attached to the second sealing member to span over the groove. In some embodiments, the first sealing member includes a cutting edge configured to puncture through the third layer when inserted into the groove of the second sealing member, and the third layer separates from the second seal member when punctured. In some embodiments, the first storage volume is open to the second storage volume when the third layer separates from the second seal member.

In some embodiments, the first and second layers define an exit opening at the second end portion of the container. In some embodiments, the apparatus further comprises a second self-sealing portion configured to seal the exit opening. The second self-sealing portion includes a first sealing member attached to the first layer at the second end portion, and a second sealing member attached to the second layer at the second end portion. In some embodiments, the third layer is attached to the second sealing member at the second end portion. In some embodiments, the third layer includes a third sealing member at the second end portion, and the third sealing member is configured to nest between the first and second sealing member when the first and second sealing member are interlocked. The third seal member is separable from the first and second sealing members when the first and second sealing members are separated to open the exit opening.

In some embodiments, the first layer, the second layer and the third layer are fixed to each other at the second end portion. In some embodiments, the second end portion includes a tear notch on a first side edge of the set of side edges and a perforated tear strip extending between the notch and a second side edge of the set of side edges. The perforated tear strip is configured to separate to detach the first layer, the second layer and the third layer from one another at the second end portion.

In some embodiments, the first layer includes one or more of fluorinated ethylene propylene (FEP), Nylon, polytetrafluoroethylene (PTFE), or polyethylene terephthalate with the addition of glycol (PETG). In some embodiments, the second layer includes one or more of fluorinated ethylene propylene (FEP), Nylon, polytetrafluoroethylene (PTFE), or polyethylene terephthalate with the addition of glycol (PETG).

In some embodiments, a method includes inserting a specimen into a first storage volume defined between a first layer of flexible material and a second layer of flexible material. The first and second layers of flexible material are fixed along a first set of side edges extending from a first end portion of the first and second layers to a central portion of the first and second layers. The specimen is inserted via an opening defined at the first end portion. The method further includes sealing the opening defined at the first end portion via a self-sealing portion of the first and second layers. The method further includes decoupling a sealing portion at the central portion of the first and second layers to open the first storage volume to a second storage volume defined between the first and second layers. The first and second layers are fixed along a second set side edges extending from the central portion of the first and second layers to a second end portion of the first and second layers. The method further includes positioning at least a portion of the specimen into the second storage volume. In some embodiments, the method further includes conveying, prior to the decoupling, a preservation fluid into the second storage volume and sealing the preservation fluid in the second storage volume.

In some embodiments, a method includes inserting a specimen into a storage volume of a flexible container, the flexible container having a first layer of flexible material, a second layer of flexible material, and a third layer of flexible material. The first, second and third layers are fixed along a set of side edges extending from a first end portion to a second end portion of the first, second and third layers, and the specimen is inserted via an opening defined between the first and second layers at the first end portion into the storage volume disposed between the first and third layers. The method includes sealing the opening defined at the first end portion between the first and second layers via a self-sealing portion of the first and second layers. In some embodiments, the sealing includes coupling a first sealing member attached to the first layer to a second sealing member attached to the second layer. In some embodiments, the method further includes conveying, prior to the separating, a preservation fluid into the second storage volume and sealing the preservation fluid in the second storage volume.

In some embodiments, the flexible container includes a second storage volume disposed between the second and third layers, the second sealing member is attached to the second layer and the third layer at the first end portion, and the coupling includes puncturing the third layer to separate the third layer from the second sealing member. In some embodiments, separating the third layer from the second sealing member opens the first storage volume to the second storage volume.

### BRIEF DESCRIPTION OF THE DRAWINGS

FIG. 1 is a schematic illustration of a container according to an embodiment.

FIG. 2 is an enlarged view of a portion of the container shown in FIG. 1.

FIG. 3 is an enlarged view of a portion of a container having a perforated opening portion, according to an embodiment.

FIG. 4 is a schematic illustration of a container according to an embodiment.

FIG. 5 is a schematic illustration of a container according to an embodiment.

FIG. 6 is a cross-sectional view of a self-sealing zipper that can be used in a container according to an embodiment.

FIG. 7 is a schematic illustration of a container according to an embodiment.

FIG. 7A is an enlarged view of the container shown in FIG. 7 with a handle configuration according to an embodiment.

FIG. 7B is an enlarged view of the container shown in FIG. 7 with another handle configuration according to an embodiment.

FIG. 8 is a schematic illustration of a container according to an embodiment.

FIG. 9 is a side cross-sectional view of the container shown in FIG. 8.

FIGS. 10A and 10B are cross-sectional views of a self-sealing portion that can be used in a container according to an embodiment.

FIG. 11A is a side cross-sectional view of the container shown in FIG. 8 in an open configuration.

FIG. 11B is a side cross-sectional view of the container shown in an open configuration according to an embodiment.

FIG. 11C is a side cross-sectional view of the container shown in FIG. 8 in a storing configuration.

FIG. 11D is a side cross-sectional view of the container shown in FIG. 8 in a retrieving configuration.

FIG. 11E is a side cross-sectional view of the container shown in FIG. 8 in a separation configuration.

FIG. 11F is a side cross-sectional view of the container shown in FIG. 8 in a retrieving configuration.

FIG. 12 is a schematic illustration of a container according to an embodiment.

FIGS. 13A and 13B are a cross-sectional views of a self-sealing portion that can be used in a container according to an embodiment.

FIG. 14A is a side cross-sectional view of the container shown in FIG. 12 in an open configuration.

FIG. 14B is a side cross-sectional view of the container shown in FIG. 12 in a storing configuration.

FIG. 14C is a side cross-sectional view of the container shown in FIG. 12 in an expanded configuration.

FIG. 14D is a side cross-sectional view of the container shown in FIG. 12 in a retrieving configuration.

FIG. 15 is a flow chart showing a method of storing a tissue specimen according to an embodiment.

FIG. 16 is a flow chart showing another method of storing a tissue specimen according to an embodiment.

### DETAILED DESCRIPTION

The embodiments described herein can advantageously be used in a wide variety of tissue recovery, handling, storage, transportation, and implantation operations. In particular, the container designs described herein can allow for a tissue specimen to be loaded and sealed at the point of loading (e.g., at a point of tissue recovery or harvest) using a self-sealing, robust container.

In some embodiments, an apparatus includes a container that is robust for storing tissue and does not require secondary containment. The container includes a self-sealing portion that allows the container to be sealed at a variety of locations (e.g., a surgical suite or the like) without specialized equipment. The self-sealing portion can include any suitable mechanism that provides a repeatable seal that limits penetration of microbes therethrough.

In some embodiments, the self-sealing portion can include a series of sealing mechanisms, such as plastic zippers, that collectively produce a tortuous path to reduce the penetration of microbes. In some embodiments, the self-sealing portion can include a tamper evident feature that provides an indication of whether the container has been reopened after initially being sealed. In some embodiments, one or more of the sealing mechanisms can include a locking or one-way member to prevent the one or more sealing mechanisms from being reopened after an initial closing of the one or more sealing mechanisms.

In some embodiments, the container can include gussets or other structural members such that the container is self-standing.

In some embodiments, the container can include a port that allows fluid communication between the storage volume and the external volume. Such an arrangement can allow for evacuation of air, ingress of preservation fluids, or the like. In some embodiments, the port may include a one-way valve. In some embodiments, the port may be coupled to tubing. In some embodiments, the tubing can be monolithically formed with the port.

As used herein, the term “about” when used in connection with a referenced numeric indication means the referenced numeric indication plus or minus up to 10% of that referenced numeric indication. For example, the language “about 50” covers the range of 45 to 55. Similarly, the language “about 5” covers the range of 4.5 to 5.5.

As used herein, the term tissue, tissue specimen or tissue graft refers to any material that is related to biological tissue. Thus, a tissue, a tissue specimen or a tissue graft can include any of a skin graft, bone tissue, fiber tissue (e.g., tendon tissue, ligament tissue, or the like), ocular tissue (e.g. corneal implants), or the like. Tissue can include a portion of tissue harvested from a donor or a structure component that includes both tissue and non-tissue material (e.g., a synthetic matrix that includes tissue therein). For example, tissue can include bone tissue that also includes bone cement or other non-tissue components. As another example, tissue, a tissue specimen or tissue graft can include bone chips including cortical bone chips, cancellous bone chips, and corticocancellous bone chips, and/or bone chips with viable bone lineage committed cells. As yet another example, tissue can include (but is not limited to) bones, skin, arteries and veins, nerves, cartilage, eyes, and birth tissues, including but not limited to amniotic membranes, amniotic fluids, placenta, etc. Tissue can also include organs or portions of an organism (e.g., enbloc; bones, tissues, muscles together, as in a whole leg vs just the fibula bone).

As used in this specification, specific words chosen to describe one or more embodiments and optional elements or features are not intended to limit the invention. For example, spatially relative terms—such as “beneath”, “below”, “lower”, “above”, “upper”, “proximal”, “distal”, and the like—may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes includes various spatial device positions and orientations.

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round”, a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

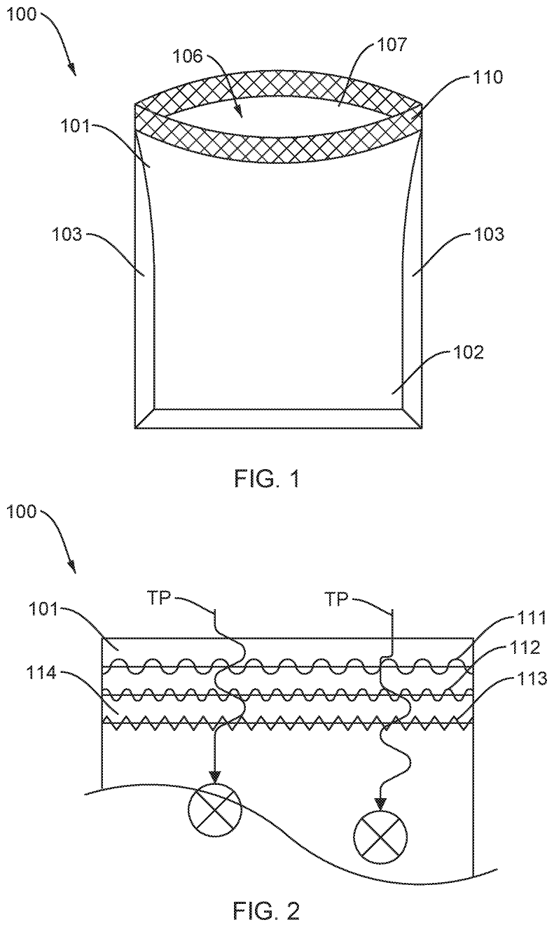

FIG. 1 is a schematic illustration of a flexible container 100 according to an embodiment. The container assembly 100 (and any of the container assemblies described herein) can be used to recover a tissue specimen (e.g., organ, bone tissue, or the like) for storage, transportation, and processing. The flexible container 100 includes a first end portion 101, a second end portion 102, and a pair of side edges 103 between the first end portion 101 and the second end portion 102. The flexible container 100 is constructed from a first layer and a second layer coupled together to define a storage volume 106. As shown in FIG. 1, when the container 100 is in the first (or opened) configuration, an edge of the first layer is spaced apart from an edge of the second layer to define an opening 107 into the storage volume 106. The opening 107 can be of any suitable size to facilitate loading of the tissue specimen (not shown), as described herein. For example, although the opening 107 is shown as extending across the full length of the first end portion 101 of the flexible container 100, in other embodiments, the opening 107 can extend across only a portion of the length of an end or a side of the flexible container 100.

The first end portion 101 of the container 100 includes a self-sealing portion 110. The self-sealing portion 110 includes one or more self-sealing mechanisms that allow the opening 107 to be closed without the need for special tools or materials (e.g., a band sealer, a heat sealer, or the like). The self-sealing portion 110 is also devoid of removable clips, bands, or wire ties. This arrangement limits variability associated with placement of such clips, bands or wire ties. Thus, the self-sealing portion 110 is also self-contained in that no external parts or equipment is needed to seal the opening 107 in a repeatable manner. The self-sealing portion 110 can include any suitable mechanism that limits the passage of microbes therethrough. For example, in some embodiments, the self-sealing portion 110 can include a self-adhesive material that, when exposed and pressed against a corresponding surface, produces a suitable seal. In other embodiments, the self-sealing portion 110 can include one or more flexible plastic zippers. Such zippers can be single-use mechanism or can be multi-use mechanism (i.e., can be resealable multiple times). In some embodiments, any of the containers described herein can include a self-sealing zipper mechanism of the types produced by Com-Pac International or any other manufacturer of self-sealing zippers. An example of a zipper profile that can be used in accordance with the embodiments described herein is shown in FIG. 6.

When sealed, the self-sealing portion 110 produces a seal that limits the transmission of microbes from an exterior volume into the inner storage volume 106 defined by the flexible container 100. For example, referring to FIG. 2, in some embodiments the self-sealing portion 110 includes a series of mechanisms that are each configured to seal the opening 107. Specifically, the self-sealing portion 110 is shown as including a first self-sealing mechanism 111, a second self-sealing mechanism 112, and a third self-sealing mechanism 113. Collectively, the self-sealing mechanisms 111, 112, 113 form a tortuous path (identified as the paths TP in FIG. 2) that reduce and/or limit the transmission of microbes from through the self-sealing portion 110 and into the storage volume 106. The self-sealing mechanisms 111, 112, 113 can be any suitable mechanism. In some embodiments, the self-sealing mechanisms 111, 112, 113 can each be the same mechanism (i.e., three mechanisms that are of the same design). For example, in some embodiments, each of the self-sealing mechanisms can be a zipper seal mechanism. In other embodiments, the self-sealing mechanisms 111, 112, 113 can be different mechanisms. For example, in some embodiments, the first self-sealing mechanism 111 can be an adhesive sealing mechanism and the second self-sealing mechanism 112 and the third self-sealing mechanism 113 can each be zipper seal mechanisms. Moreover, although shown as including three self-sealing mechanisms in other embodiments, the container 100 can include any number of sealing mechanisms (e.g., a single self-sealing mechanism, two mechanisms, four mechanisms, five mechanisms, etc.).

In some embodiments, the self-sealing portion 110 includes one or more self-sealing mechanisms that can be repeatably opened and closed. Moreover, in some embodiments, all or a portion of the self-sealing portion 110 can include an indicator to identify when the self-sealing portion 110 is fully closed, or to identify when the self-sealing portion 510 has been opened or tampered with after a first instance of closing. Such an indicator can be a change in color, the creation of a “sealing line” (or other visual indicator) that occurs only upon satisfactory sealing, or the like. In other embodiments, the self-sealing portion 110 includes one or more self-sealing mechanisms that are designed as single-use mechanisms. This prevents a tissue sample from being accessed after it has been sealed in the container 100. Moreover, in some embodiments, the self-sealing portion 110 can include an indicator to identify when the self-sealing portion 110 has been opened (after an initial instance of having been fully sealed). Similarly stated, in some embodiments, the self-sealing portion 110 can include a tamper indicator.

The side edges 103 and the second end portion 102 can be joined together in any suitable manner. For example, in some embodiments, the side edges can be joined by a weld, heat bonding, or other joining technique that produces a linear joint with substantially no thickness. In other embodiments, the side edges 103 and the second end portion 102 can be joined together to form a seal that has a length and a width. In this manner, the side edges and/or the second end portion can have a “spine” that provides structural support for the edges of the bag. For example, in some embodiments, the side edges 103 can include a gusset, support, pleat or the like that strengthens the side edges and allows the container 100 to remain in an opened configuration by default. In other embodiments, the side edges 103 can include a gusset, support, pleat or the like that allows the container 100 to be a self-standing container.

The flexible container 100 and any of the containers described herein can be constructed of any material suitable for storing tissue samples that is robust and resistant to damage. For example, in some embodiments, material from which all or portions of the flexible container 100 are constructed can include fluorinated ethylene propylene (FEP), Nylon, polytetrafluoroethylene (PTFE), polyethylene terephthalate with the addition of glycol (PETG), or any other flexible polymer. In some embodiments, all or portions of the flexible container 100 can be constructed from a thin, peelable film, such as, for example, a heat seal-coated (HSC) material, a polyethylene material, a polyvinyl chloride (PVC) material, a polyamide material, a polyester-based material, or any combination of such materials. In some embodiments, the flexible container 100 can be constructed from a series of laminates constructed from multiple different materials.

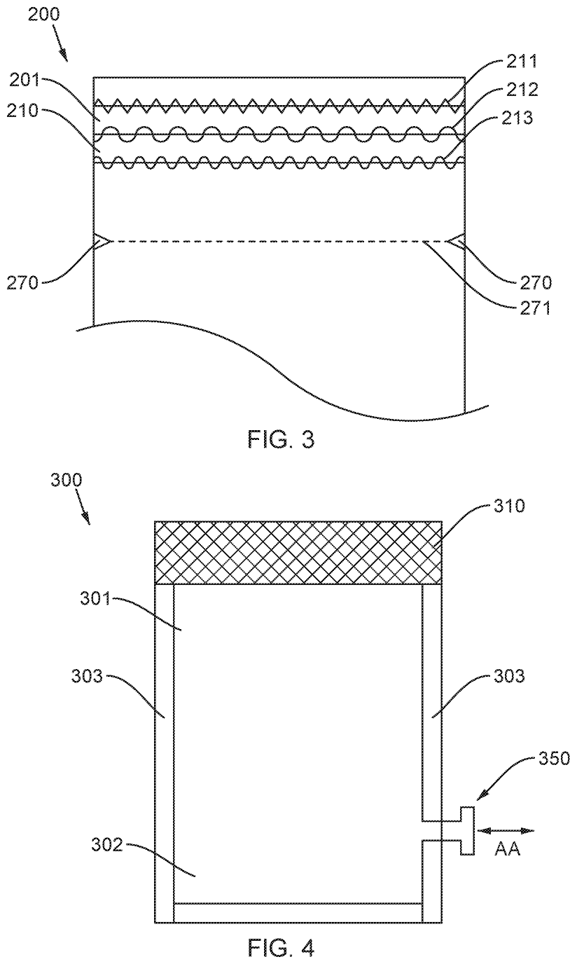

In some embodiments, a container can be configured for only a single use and can include a self-sealing portion that has one or more mechanism that “lock” or otherwise prevent re-opening of the container. In such embodiments, the container can include one or more mechanisms to allow the container to be easily opened for removal of the tissue therein. For example, FIG. 3 shows a portion of a container 200 having a self-sealing portion 210, according to an embodiment. The self-sealing portion 210 includes a series of mechanisms that are each configured to seal an opening into the container (not shown) at a first end portion 201 extending between side edges 203. Specifically, the self-sealing portion 210 is shown as including a first self-sealing mechanism 211, a second self-sealing mechanism 212, and a third self-sealing mechanism 213. Collectively, the self-sealing mechanisms 211, 212, 213 form a tortuous path that reduces and/or limits the transmission of microbes from through the self-sealing portion 210 and into the storage volume of the container. The self-sealing mechanisms 211, 212, 213 can be any suitable mechanism that is configured for one use. Similarly stated, the self-sealing mechanisms 211, 212, 213 are configured to limit or prevent reopening of the container 200 after initially being sealed. In some embodiments, the self-sealing mechanisms 211, 212, 213 can each be the same mechanism (i.e., three mechanisms that are of the same design). For example, in some embodiments, each of the self-sealing mechanisms can be a zipper seal mechanism. In other embodiments, the self-sealing mechanisms 211, 212, 213 can be different mechanisms. For example, in some embodiments, the first self-sealing mechanism 211 can be an adhesive sealing mechanism and the second self-sealing mechanism 212 and the third self-sealing mechanism 213 can each be zipper seal mechanisms. Moreover, although shown as including three self-sealing mechanisms in other embodiments, the container 200 can include any number of sealing mechanisms (e.g., a single self-sealing mechanism, two mechanisms, four mechanisms, five mechanisms, etc.).

To facilitate opening of the container 200, the container also includes an opening portion 271. The opening portion can be a perforated portion and can include tear notches 270 to facilitate easy opening of the container. Although the tear notches 270 are illustrated as adjacent to and on a same side as the opening portion 271, the tear notches 270 may be provided on a side of the container 200 opposite from the opening portion 271. In some embodiments, the tear notches 270 may be provided on one or both side edges 203. In other embodiments, the container 200 (or any of the containers described herein) can include one or more peelable seals to facilitate opening the container after being sealed close via the self-sealing portion 210. For example, in some embodiments, one of the side edges of the container can include a peelable seal that hermetically seals the storage volume. The peelable seal can be similar to the seals shown and described in U.S. application Ser. No. 16/460,920, filed Jul. 2, 2019, entitled “Sample Container with Peelable Seal and Access Port,” which is incorporated herein by reference in its entirety. The peelable seal can be configured to have any suitable failure (or peel) mechanism, and can be of any suitable peel strength. For example, in some embodiments, any of the containers described herein can include a peelable seal 120 that is an adhesive-based seal in which an adhesive layer pulls back from one of a first layer or a second layer when the first layer is peeled apart from the second layer. In other embodiments, a peelable seal can be a cohesive seal in which an adhesive layer or intermediate layer fails within itself when the first layer is peeled apart from the second layer. The peelable seal can be produced by any suitable mechanism as described herein, such as, for example, by a heat sealing operation. The inclusion of the perforated opening portion 271 or a peelable seal also eliminates the need for extra tools for opening the container 200 during use.

In some embodiments, any of the containers described herein can include a port that can selectively place the storage volume in fluid communication with an exterior volume. This arrangement can allow air to be purged from the storage volume during or after the sealing of the self-sealing portion. The port can also allow for contents (e.g., storage fluid, preservation fluid, wash solutions, or the like) to be placed into the container after the self-sealing portion has been sealed. For example, FIG. 4 is a schematic illustration of a flexible container 300 according to an embodiment. The container assembly 300 (and any of the container assemblies described herein) can be used to recover a tissue specimen (e.g., organ, bone tissue, or the like) for storage, transportation, and processing. The flexible container 300 includes a first end portion 301, a second end portion 302, and a pair of side edges 303 between the first end portion 301 and the second end portion 302. The flexible container 300 is constructed from a first layer and a second layer coupled together to define a storage volume.

The first end portion 301 of the container 300 includes a self-sealing portion 310. The self-sealing portion 310 includes one or more self-sealing mechanisms that allow an opening (not shown) to be closed without the need for special tools or materials (e.g., a band sealer, a heat sealer, or the like). The self-sealing portion 310 is also devoid of removable clips, bands, or wire ties. This arrangement limits variability associated with placement of such clips, bands or wire ties. Thus, the self-sealing portion 310 is also self-contained in that no external parts or equipment is needed to seal the opening 307 in a repeatable manner. The self-sealing portion 310 can include any suitable mechanism that limits the passage of microbes therethrough. For example, in some embodiments, the self-sealing portion 310 can include a self-adhesive material that, when exposed and pressed against a corresponding surface, produces a suitable seal. In other embodiments, the self-sealing portion 310 can include one or more flexible plastic zippers. Such zippers can be single-use mechanism or can be multi-use mechanism (i.e., can be resealable multiple times). In some embodiments, any of the containers described herein can include a self-sealing zipper mechanism of the types produced by Com-Pac International or any other manufacturer of self-sealing zippers. An example of a zipper profile that can be used in accordance with the embodiments described herein is shown in FIG. 6.

The second end portion 302 includes a port 350 configured to allow fluid communication (as shown by the arrow AA in FIG. 4) between a volume outside of the container assembly 300 and the storage volume 306. Thus, the port 350 can be used to provide access to the storage volume 306 and the tissue specimen (not shown in FIG. 4) after the first end portion 301 has been sealed closed. In this manner, the tissue specimen can be treated with a preservation fluid or other material after being sealed into the container assembly 300. The port 350 can also be coupled to a vacuum source to evacuate the storage volume for storage of the tissue specimen. Moreover, during a surgical procedure, the port 350 can allow for inflow of rehydration fluid.

The port 350 can be any suitable port that selectively provides fluid communication to the storage volume. For example, the port 350 can include a tube, a valve, and/or a cap. In some embodiments, the port may include a one-way valve. In some embodiments, the port may be coupled to tubing. In some embodiments, the tubing can be monolithically formed with the port. In some embodiments, the port 350 can be a needle-free port. In some embodiments, the port 350 can be a swabable connector. Similarly stated in some embodiments, the port 350 can have external surfaces and can be devoid of recesses or crevices such that the port 350 can be easily wiped or “swabbed” to maintain sterility during use. In some embodiments, the port 350 can include any of the barbed, swabable valves produced by the Halkey-Roberts Corporation, such as the 2455 series of swabable valves. Although the port 350 is shown as being coupled at the second end portion 302 of the flexible container 300, in other embodiments, the port 350 (and any of the ports described herein) can be coupled at any location and to any portion of the flexible container 300.

In some embodiments, the container 100 (and any of the containers described herein) can be used to store the tissue specimen for transportation, later processing, and/or later use. For example, in some embodiments a method of use includes placing the tissue specimen into the storage volume 106 of the flexible container 100. After the tissue specimen is within the storage volume 106, the self-sealing portion 110 is then sealed closed. This can include, for example, manipulating the first self-sealing mechanism 111, the second self-sealing mechanism 112, and the third self-sealing mechanism 113 to form a hermetic barrier through which microbes cannot pass. In some embodiments, the self-sealing portion 110 can be sealed via a single operation. For example, in some embodiments, the each of the self-sealing mechanisms can be a zipper mechanism, each of which are coupled to a single slider (or actuator). The self-sealing portion 110 can be sealed by moving the single slider to close the container.

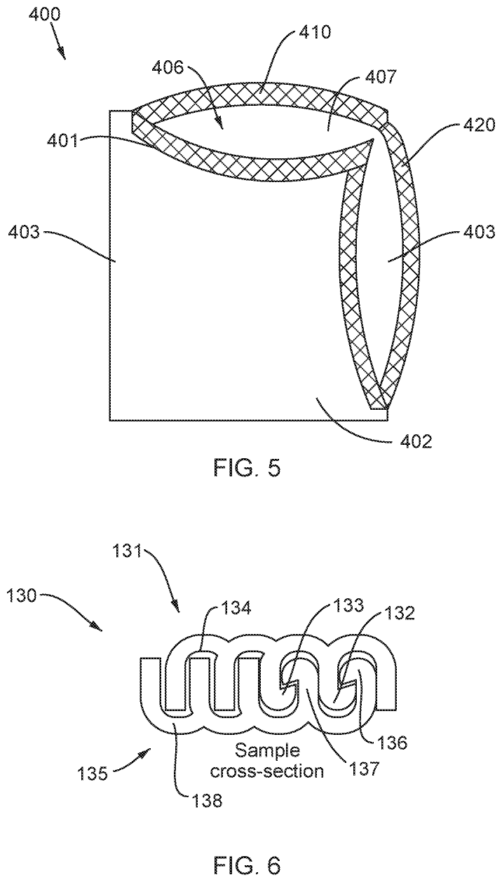

Although the self-sealing portion 110 is shown as being along only the top edge of the container 100, in other embodiments, any of the containers described herein can include a self-sealing portion that extends along multiple edges. For example, FIG. 5 is a schematic illustration of a flexible container 400 according to an embodiment. The container assembly 400 (and any of the container assemblies described herein) can be used to recover a tissue specimen (e.g., organ, bone tissue, or the like) for storage, transportation, and processing. The flexible container 400 includes a first end portion 401, a second end portion 402, and a pair of side edges 403 between the first end portion 401 and the second end portion 402. The flexible container 400 is constructed from a first layer and a second layer coupled together to define a storage volume 406. As shown in FIG. 5, when the container 400 is in the first (or opened) configuration, an edge of the first layer is spaced apart from an edge of the second layer to define an opening 407 into the storage volume 406. This arrangement can allow the container 400 to opened like a “flap” by folding one layer outward to expose the storage volume 406. The opening 407 can be of any suitable size to facilitate loading of the tissue specimen (not shown), as described herein. For example, although the opening 407 is shown as extending across the full length of the first end portion 401 and down along one side edge 403 of the flexible container 400, in other embodiments, the opening 407 can extend across only a portion of the length of an end or a side of the flexible container 400.

The first end portion 401 of the container 400 includes a first self-sealing portion 410 and one of the side edges 403 includes a second self-sealing portion 420. The first self-sealing portion 410 and the second self-sealing portion 420 can each include one or more self-sealing mechanisms that allow the opening 407 to be closed without the need for special tools or materials (e.g., a band sealer, a heat sealer, or the like). The self-sealing portions 410, 420 are also devoid of removable clips, bands, or wire ties. This arrangement limits variability associated with placement of such clips, bands or wire ties. Thus, the self-sealing portions 410, 420 are also self-contained in that no external parts or equipment is needed to seal the opening 407 in a repeatable manner. The self-sealing portions 410, 420 can include any suitable mechanism as described herein. Moreover, the container 400 can include any number of sealing mechanisms (e.g., a single self-sealing mechanism, two mechanisms, four mechanisms, five mechanisms, etc.).

In some embodiments, the self-sealing portions 410, 420 include one or more self-sealing mechanisms that can be repeatably opened and closed. Moreover, in some embodiments, all or a portion of the self-sealing portions 410, 420 can include an indicator to identify when the self-sealing portions 410, 420 are fully closed. Such an indicator can be a change in color, the creation of a “sealing line” (or other visual indicator) that occurs only upon satisfactory sealing, or the like. In other embodiments, the self-sealing portions 410, 420 include one or more self-sealing mechanisms that are designed as single-use mechanisms. This prevents a tissue sample from being accessed after it has been sealed in the container 400. Moreover, in some embodiments, the self-sealing portions 410, 420 can include an indicator to identify when the self-sealing portions 410, 420 has been opened (after an initial instance of having been fully sealed). Similarly stated, in some embodiments, the self-sealing portions 410, 420 can include a tamper indicator.

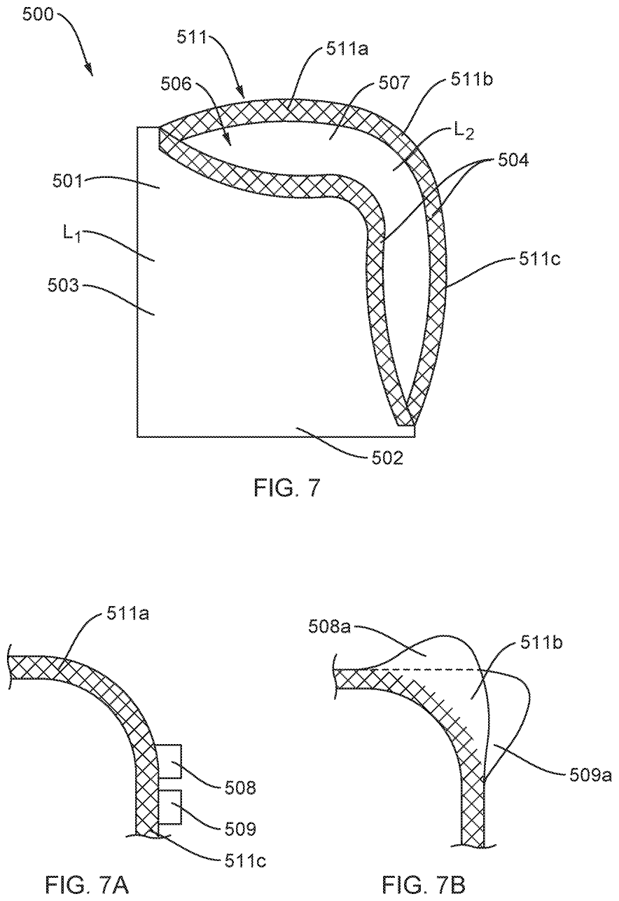

FIG. 7 is a schematic illustration of a flexible container 500 according to an embodiment. The container assembly 500 (and any of the container assemblies described herein) can be used to recover a tissue specimen (e.g., organ, bone tissue, or the like) for storage, transportation, and processing. The flexible container 500 includes a first end portion 501, a second end portion 502, a first side portion 503 and a second side portion 504. The first side portion 503 and the second side portion 504 each extend between the first end portion 501 and the second end portion 502. The flexible container 500 is constructed from a first layer L₁ and a second layer L₂ coupled together to define a storage volume 506. As shown in FIG. 7, when the container 500 is in the first (or opened) configuration, an edge of the of the first layer L₁ is spaced apart from an edge of the second layer L₂ to define an opening 507 into the storage volume 506. In some embodiments, the edge includes at least one of the first end portion 501 or the second end portion 502 and one of the first side portion 503 or the second side portion 504. This arrangement can allow the container 500 to opened like a “flap” by folding one layer outward to expose the storage volume 506. In some embodiments, each of the layers may include a handle portion to assist an operator in opening and/or maintaining the container 500 in an open position. For example, as shown in FIG. 7A, the container may include a first handle portion 508 and a second handle portion 509, the second handle portion 509 being laterally offset from the first handle portion 508. In some embodiments, one of the handle portions may be attached to the first layer L₁ and the other handle portion may be attached to the second layer L₂ (the first layer L₁ and the second layer L₂ forming the container 500 at the first end portion 501, the first side portion 503, or the second side portions 504). In some embodiments, the first and second handle portions 508, 509 are attached to the first layer L₁ and the second layer L₂, respectively, via adhesives, co-molding, or other bonding techniques. In some embodiments, as shown in FIG. 7B, the first layer L₁ and second layer L₂ have asymmetrical perimeter footprints and the first and second handle portions 508a, 509b are monolithically formed with the first layer L₁ and second layer L₂, respectively.

The opening 507 can be of any suitable size to facilitate loading of the tissue specimen (not shown), as described herein. For example, although the opening 507 is shown as extending across the full length of the first end portion 501 and down along the second side portion 504 of the flexible container 500, in other embodiments, the opening 507 can extend across only a portion of the length of an end portion or a side portion of the flexible container 500. The first end portion 501 and the second side portion 504 includes a self-sealing portion 510 that extends an entire length of the opening 507. In some embodiments, the self-sealing portion 510 can include a sealing mechanism 511 with a first linear segment section 511a, a second linear segment section 511b, and a curved segment section 511c, the curved segment section 511c being disposed between the first linear segment section 511a and the second linear segment section 511b. In some embodiments, as shown in FIG. 7B, the curved segment section 511c is disposed between the first and second handle portions 508a, 509a.

The self-sealing portion 510 can include one or more self-sealing mechanisms that allow the opening 507 to be closed without the need for special tools or materials (e.g., a band sealer, a heat sealer, or the like). The self-sealing portion 510 are also devoid of removable clips, bands, or wire ties. This arrangement limits variability associated with placement of such clips, bands or wire ties. Thus, the self-sealing portion 510 is also self-contained in that no external parts or equipment is needed to seal the opening 507 in a repeatable manner. The self-sealing portion 510 can include any suitable mechanism as described herein. Moreover, the container 500 can include any number of sealing mechanisms (e.g., a single self-sealing mechanism, two mechanisms, four mechanisms, five mechanisms, etc.).

In some embodiments, the self-sealing portion 510 can include one or more self-sealing mechanisms that can be repeatably opened and closed. Moreover, in some embodiments, all or a portion of the self-sealing portion 510 can include an indicator to identify when the self-sealing portion 510 are fully closed, or to identify when the self-sealing portion 510 has been opened or tampered with after a first instance of closing. Such an indicator can be a change in color, the creation of a “sealing line” (or other visual indicator) that occurs only upon satisfactory sealing, or the like. In other embodiments, the self-sealing portion 510 can include one or more self-sealing mechanisms that are designed as single-use mechanisms. This prevents a tissue sample from being accessed after it has been sealed in the container 500. Moreover, in some embodiments, the self-sealing portion 510 can include an indicator to identify when the self-sealing portion 510 has been opened (after an initial instance of having been fully sealed). Similarly stated, in some embodiments, the self-sealing portion 510 can include a tamper indicator.

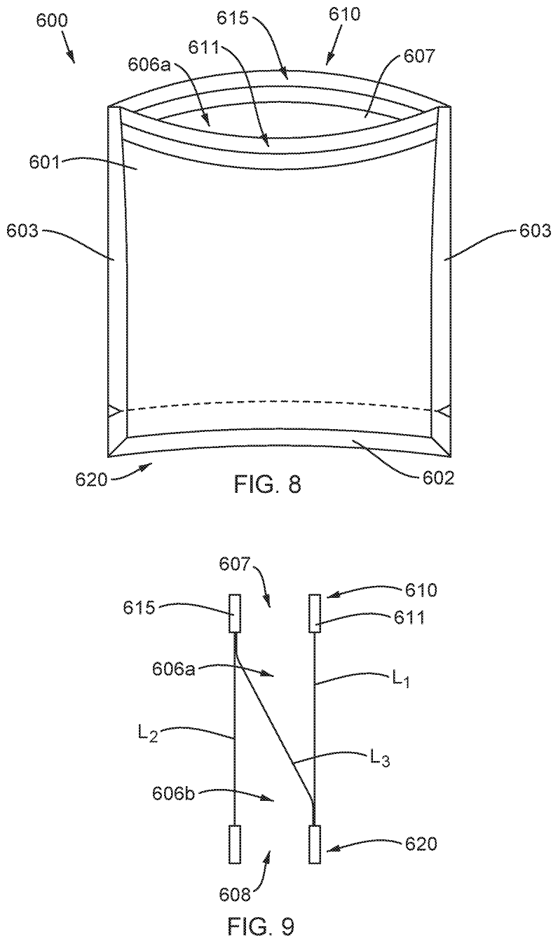

Although the container 500 is shown as including a first layer and a second layer, in other embodiments, a flexible container can include any number of layers and can include any number of different internal partitions to facilitate storage, easy removal, and the inclusion of fluids (e.g., preservation fluids) to facilitate tissue handling. For example, FIGS. 8-11F are various schematic illustrations of a flexible container 600 according to an embodiment. The container assembly 600 (and any of the container assemblies described herein) can be used to recover a tissue specimen (e.g., organ, bone tissue, or the like) for storage, transportation, and processing. The flexible container 600 includes a first end portion 601, a second end portion 602, and a pair of side edges 603 between the first end portion 601 and the second end portion 602. As shown in FIG. 9, the flexible container 600 is constructed from a first layer L₁, a second layer L₂, and a third layer L₃. When the layers are coupled together, the flexible container 600 defines a first storage volume 606a between the first layer L₁ and third layer L₃ and a second storage volume 606b between the third layer L₃ and the second layer L₂. As shown in FIG. 9, when the container 600 is in the first (or open entry) configuration, an edge of the first layer L₁ is spaced apart from an edge of the second layer L₂ to define an entry opening 607 into the first storage volume 606a. The entry opening 607 can be of any suitable size to facilitate loading of the tissue specimen S, as described herein. For example, although the entry opening 607 is shown as extending across the full length of the first end portion 601 of the flexible container 600, in other embodiments, the entry opening 607 can extend across only a portion of the length of an end or a side of the flexible container 600.

The first end portion 601 of the flexible container 600 includes a first self-sealing portion 610 to join the first layer L₁ to the second layer L₂ at the entry opening 607. The first self-sealing portion 610 includes one or more self-sealing mechanisms that allow the entry opening 607 to be closed without the need for special tools or materials (e.g., a band sealer, a heat sealer, or the like). The first self-sealing portion 610 is also devoid of removable clips, bands, or wire ties. This arrangement limits variability associated with placement of such clips, bands or wire ties. Thus, the first self-sealing portion 610 is also self-contained in that no external parts or equipment is needed to seal the entry opening 607 in a repeatable manner. The first self-sealing portion 610 can include any suitable mechanism that limits the passage of microbes therethrough. For example, in some embodiments, the first self-sealing portion 610 can include a self-adhesive material that, when exposed and pressed against a corresponding surface, produces a suitable seal. In other embodiments, the first self-sealing portion 610 can include one or more flexible plastic zippers. Such zippers can be single-use mechanism or can be multi-use mechanism (i.e., can be resealable multiple times). In some embodiments, any of the containers described herein can include a self-sealing zipper mechanism of the types produced by Com-Pac International or any other manufacturer of self-sealing zippers. An example of a zipper profile that can be used in accordance with the embodiments described herein is shown in FIG. 6.

In some embodiments, as shown in FIGS. 9 to 11, the first self-sealing portion 610 includes a first sealing member 611 joined to or formed with the first layer L₁ and a second sealing member 615 joined to or formed with the second layer L₂ and the third layer L₃. The first sealing member 611 is configured to engage and interlock with the second sealing member 615. In some embodiments, the first sealing member 611 includes one or more of a first hook and groove channel 612a, a second hook and groove channel 612b, a rib 613, and a cutting edge 614. The second sealing member 615 includes one or more of a first hook and groove channel 616a, a second hook and groove channel 616b, a first groove 617, and a second groove 618. The first hook and groove channel 612a is configured to be inserted into and engage with the first hook and groove channel 616a. The second hook and groove channel 612b is configured to be inserted into and engage with the second hook and groove channel 616b. The rib 613 is configured to be inserted into the first groove 617, and the cutting edge 614 is configured to be inserted into the second groove 618.

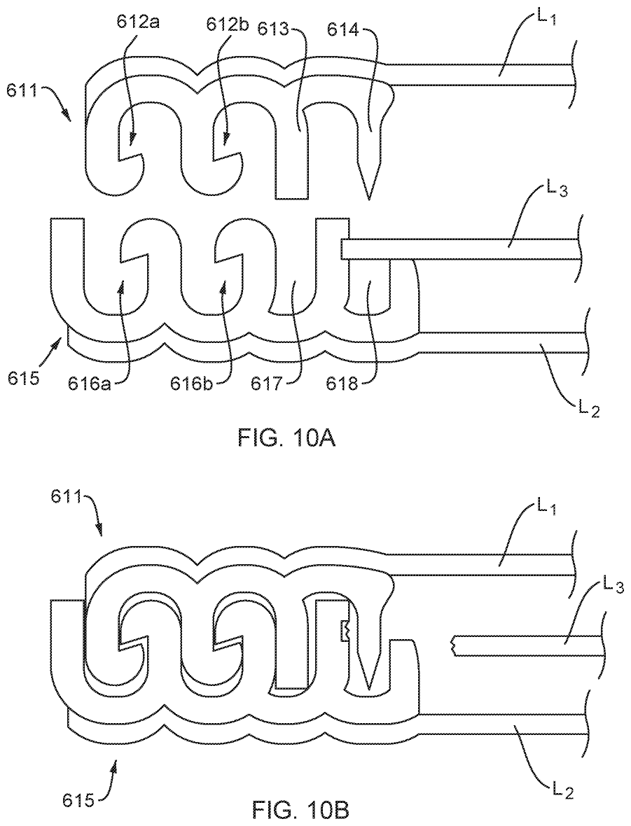

In some embodiments, as shown in greater detail in FIGS. 10A and 10B, the first layer L₁ is secured to the first sealing member 611 on a side opposite of the openings for the first hook and groove channel 612a and the second hook and groove channel 612b. The second layer L₂ is secured to the second sealing member 615 on a side opposite of the openings for the first hook and groove channel 616a and the second hook and groove channel 616b. The third layer L₃ is secured to a same side of the opening of at least one of the first groove 617 and the second groove 618. In some embodiments, the third layer L₃ is secured to a wall of the second groove 618, such that a portion of the third layer L₃ bridges over the second groove 618. In some embodiments, as the first sealing member 611 is secured and interlocked with the second sealing member 616, the cutting edge 614 is operable to puncture through the third layer L₃ as it enters into the second groove 618, thereby separating the third layer L₃ from the second layer L₂ at or near the first end portion 601. By separating the third layer L₃ from the second layer L₂, the first storage volume 606a can be open to, merged or combined with the second storage volume 606b to enable the specimen S to move between the volumes and/or enable other fluids or materials stored within the second storage volume 606b to mix with the specimen S. For example, at least one of the first storage volume 606a or the second storage volume 606b can be coupled to a port, as described above, to introduce fluids or other materials into one or both of the volumes 606a, 606b.

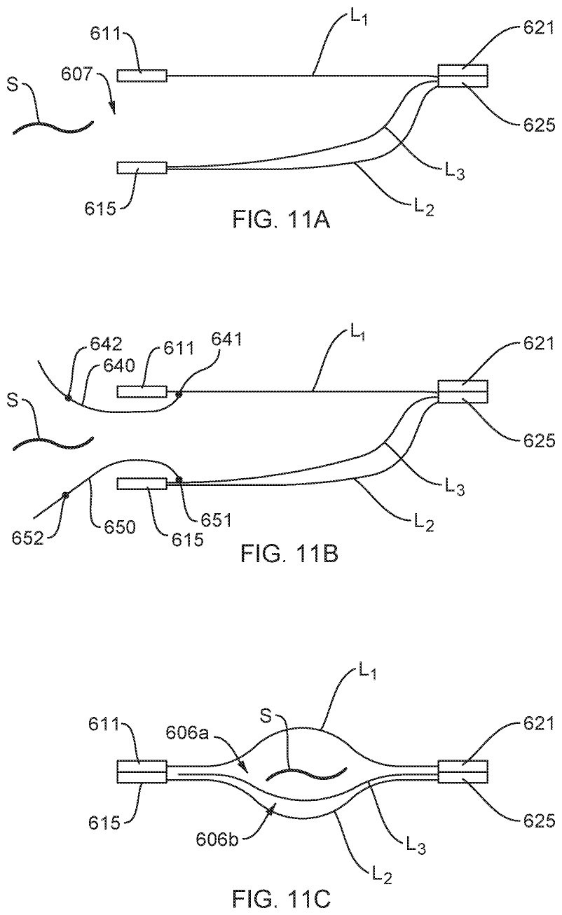

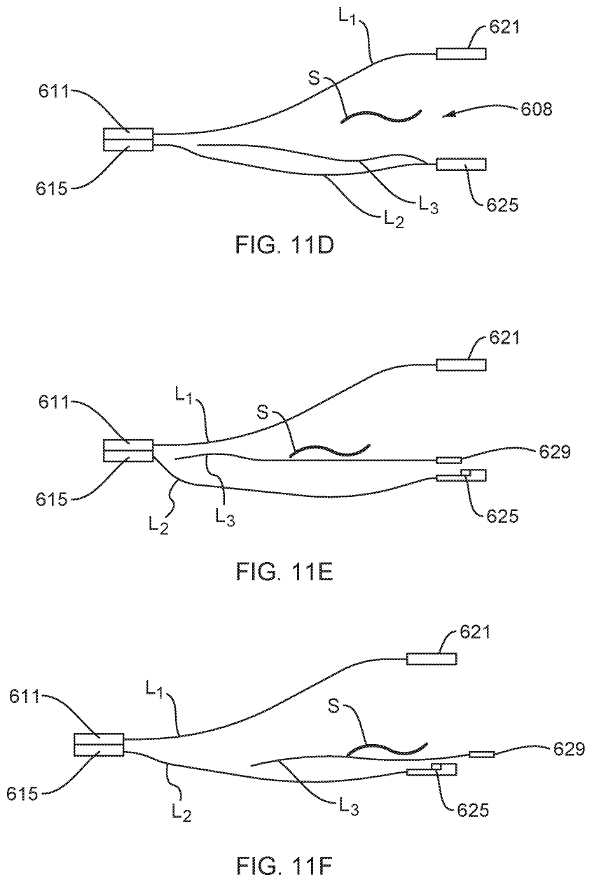

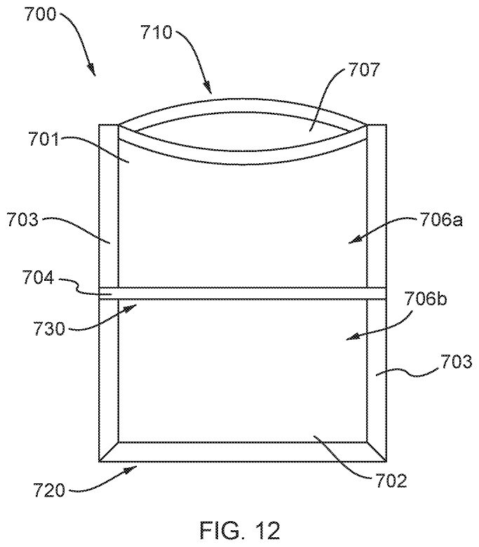

In some embodiments, the second end portion 602 of the flexible container 600 can include a second sealing portion 620. The second sealing portion 620 can be any type of self-sealing portion described herein, a sealed perforated tear strip that can include tear notches 670, a peelable seal, or any combination thereof. In some embodiments, a tear notch 670 can be provided on one or both of the side edges 603. As shown in FIG. 11A when the flexible container 600 is in a first (or receiving) configuration, the first layer L₁ and the second layer L₂ are separated at the first end portion 601 and the second layer L₂ and the third layer L₃ are joined at the first end portion 601. The first layer L₁, the second layer L₂ and the third layer L₃ are joined together at the second end portion 602. In the first configuration, the entry opening 607 is formed at the first end portion 601 between the first layer L₁ and the second layer L₂ and is bounded by the first self-sealing portion 610. In this first configuration, a tissue specimen S can be inserted into the first storage volume 606a.

In some embodiments, as shown in FIG. 11B, the flexible container 600 can further include a first flap portion 640 and a second flap portion 650 to prevent the tissue specimen S from contacting the first and second sealing members 611, 612. In some instances, the first and second sealing members 611, 612 may have rough or uneven surfaces (e.g., hook and groove channels) that may catch on or scrape the tissue specimen S during loading of the container, causing damage to the tissue specimen S and/or causing the first and second sealing members 611, 612 to clog, preventing proper closure and sealing of the first and second sealing members 611, 612. In some embodiment, the flap portions 640, 650 can include a set of perforations 641, 651 internally located relative to the first and second sealing members 611, 612. The set of perforations 642, 652 can enable the flap portions 640, 650 to be detached from the flexible container 600 after the specimen S has been inserted into the first storage volume 606a. Additionally, or alternatively, the flap portions 640, 650 can include a set of perforations 642, 652, disposed externally relative to the first and second sealing members 611, 612 when the flap portions 640, 650 extend out of the entry opening 607. The set of perforations 642, 652 can enable portions of the flap portions 640, 650 to be detached from the flexible container 600 after the specimen has been inserted into the first storage volume 606a and prior to closing the first and second sealing members 611, 612. The remaining length of flap portions 640, 650 can be tucked past the entry opening 607 and into the first storage volume 606a prior to closing the first and seal members 611, 612. In some embodiments, the pair of flap portions 640, 650 can be formed as a continuous sleeve or funnel that encircles the entry opening 607.

Although the protective flaps are described as being a first flap portion 640 and a second flap portion 650, in other embodiments, any of the containers described herein can include a protective flap that is monolithically constructed. For example, in some embodiments, the first flap portion 640 and the second flap portion 650 are portions of a single protective structure.

When the flexible container 600 is in a second (or storage) configuration, as shown in FIG. 11C, the first sealing member 611 and the second sealing member 612 are joined together and interlocked such that the first layer L₁ is sealed with the second layer L₂ at the first end portion 601. At the same time, the third layer L₃ is detached from the second sealing member 612 such that the first storage volume 606a and the second storage volume 606b is combined. In some embodiments, the second storage volume 606b can be prefilled with preservation fluid such that after tissue specimen S is inserted into the first storage volume 606a and the first self-sealing portion 610 is closed, the third layer L₃ is detached or punctured to introduce the preservation fluid from the second storage volume 606b into the first storage volume 606a. By providing the preservation fluid in the second storage volume 606b, separate steps for connecting and introducing preservation fluid into the container is eliminated, thereby removing a step for potential user error. The second storage volume 606b with prefilled preservation fluid can also minimize the time between placement of a specimen in the container and introduction of the preservation fluid with the specimen, thereby improving preservation of the harvested tissue. The prefilled volume of preservation fluid can enable the flexible container 600 to be used in remote or off-site locations, and can minimize introduction of foreign matter into the flexible container 600 by eliminating separate steps of connecting and filling the container with an external source of preservation fluid after the specimen has been placed in the container. The flexible container 600 can be used, for example, in the field for prompt on-site harvesting of tissues from a donor, and the on-site harvesting can take place outside of a hospital or laboratory setting.

When the flexible container 600 is in a third (or retrieving) configuration, as shown in FIG. 11D, the second sealing portion 620 can be opened separating one or more of the first layer L₁, the second layer L₂ and the third layer L₃ from one another to form an exit opening 608 into the first and second storage volumes 606a, 606b. In this third configuration, the tissue specimen S can be removed from the flexible container 600 via the exit opening 608. In some embodiments, as shown in FIGS. 11E and 11F, the second sealing portion 620 includes a first sealing member 621, a second sealing member 625, and a third sealing member 629, the third sealing member 629 being nested between the first sealing member 621 and the second sealing member 625. The first sealing member 621 is attached to the first layer L₁ at the second end portion 602, the second sealing member 625 is attached to the second layer L₂ at the second end portion 602, and the third sealing member 629 is attached to the third layer L₃ at the second end portion 602. As the first sealing member 621 is separated from the second sealing member 625, the third sealing member 629 can be separated from the first sealing member 621 and second sealing member 625 such that the specimen S can be removed from the flexible container 600 together with the third layer L₃, the third layer L₃ serving as a conveyance member to minimize direct contact and damage to the specimen S as it is being removed from the flexible container 600.

FIG. 12 is a schematic illustration of a flexible container 700 according to an embodiment. The container assembly 700 (and any of the container assemblies described herein) can be used to recover a tissue specimen (e.g., organ, bone tissue, or the like) for storage, transportation, and processing. The flexible container 700 includes a first end portion 701, a second end portion 702, and a pair of side edges 703 between the first end portion 701 and the second end portion 702. The flexible container 700 includes a central portion 704 disposed between the first end portion 701 and the second end portion 702. The central portion 704 can subdivide an interior of the flexible container 700 into a first storage volume 706a and a second storage volume 706b.

The flexible container 700 is constructed from a first layer L₁ and a second layer L₂. When the flexible container 700 is open at the first end portion 701, an edge of the first layer L₁ is spaced apart from an edge of the second layer L₂ to define an opening 707 into the first storage volume 706a. The opening 707 can be of any suitable size to facilitate loading of the tissue specimen S, as described herein. For example, although the opening 707 is shown as extending across the full length of the first end portion 701 of the flexible container 700, in other embodiments, the opening 707 can extend across only a portion of the length of an end or a side of the flexible container 700.

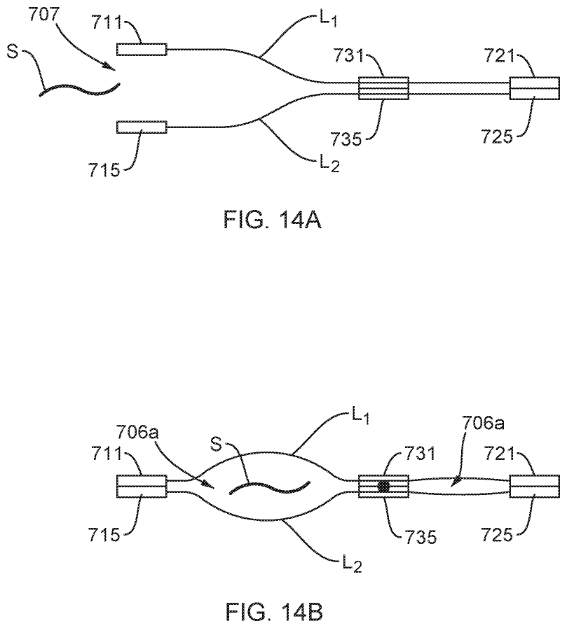

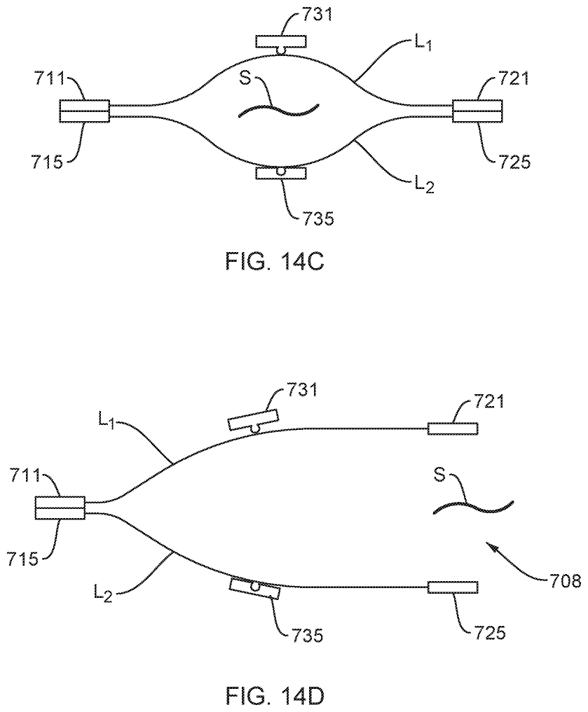

The first end portion 701 of the flexible container 700 includes a first self-sealing portion 710 to join the first layer L₁ to the second layer L₂ at the opening 707. The first self-sealing portion 710 can include one or more self-sealing mechanisms described above and can be sealed without the need for special tools or materials (e.g., a band sealer, a heat sealer, or the like). For example, the first self-sealing portion 710 can include a first seal member 711 and a second seal member 715. The second end portion 702 can also be one or more self-sealing mechanisms 720, one or more peelable seals, and/or perforated tear strip for opening the second end portion 702. For example, as shown in FIGS. 14A-14D, the second self-sealing portion 720 can include a first seal member 721 and a second seal member 725. The central portion 704 includes a first seal member 731 attached to the first layer L₁ on an exterior surface of the flexible container 700, and the central portion 704 includes a second seal member 735 attached to the second layer L₂ on an exterior surface of the flexible container 700.

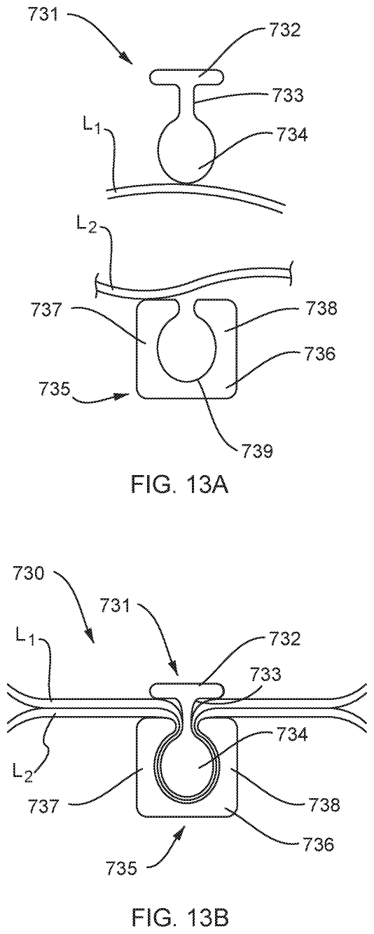

For example, as shown in FIG. 13A, the first seal member 731 can includes a base portion 732, a neck portion 733 and a head portion 734. The first seal member 731 can extend a width of the flexible container 700 between the pair of side edges 703. The first seal member 731 can be secured to the first layer L₁ via the head portion 734. The head portion 734 can be monolithically formed with the first layer L₁, co-molded with the first layer L₁, or joined to the first layer L₁ via adhesives or other bonding techniques. The first layer L₁ can be secured to only to a segment of the head portion 734 such that the first layer L₁ is operable to conform around the head portion 734 or to any object that may be placed within the flexible container 700.

The second seal member 735 can includes a base portion 736, a first shoulder 737, a second shoulder 738, and a groove 739 defined between the first and second shoulders 737, 738. The second seal member 735 can extend a width of the flexible container 700 between the pair of side edges 703. In some embodiments, the second seal member 735 can be secured to the second layer L₂ via either or both of the first shoulder 737 and the second shoulder 738. The first shoulder 737 or the second shoulder 738 secured to the second layer L₂ can be monolithically formed with the second layer L₂, co-molded with the second layer L₂, or joined to the second layer L₂ via adhesives or other bonding techniques. The second layer L₂ can be secured to one of the first or second shoulders 737, 738 such that the second layer L₂ is operable to be taken up within the groove 739 or to any object that may be placed within the flexible container 700.

In a coupled configuration, as shown in FIG. 13B, the central portion 704 of the flexible container 700 can be joined together via the first seal member 731 and the second seal member 735. In the coupled configuration, the head portion 734 is inserted into the groove 739 such that the first layer L₁ and the second layer L₂ are interposed therebetween. The first seal member 731 and the second seal member 735 are secured to the first layer L₁ and the second layer L₂, respectively, such that the layers can be taken up between the head portion 734 and the groove 739.

With reference to FIGS. 14A-14D, various configurations and use of the flexible container 700 will now be described. In a first (or open entry) configuration the first layer L₁ and the second layer L₂ are separated at the first end portion 701 and the first layer L₁ and the second layer L₂ are joined together at the central portion 704 and the second end portion 702. In this first configuration, a tissue specimen S can be inserted into the first storage volume 706a via an entry opening 707.

When the flexible container 700 is in a second (or storing) configuration, as shown in FIG. 14B, the first self-sealing portion 710 is joined together and interlocked such that the first layer L₁ is sealed with the second layer L₂, at the first end portion 701. The specimen S is then sealed within the first storage volume 706a. In some embodiments, the flexible container 700 can then be placed in a third (or expanded) configuration by separating the first layer L₁ from the second layer L₂ at the central portion 704 to combine the first storage volume 706a and the second storage volume 706b. As shown in FIG. 14C, the central portion 704 can be separated by applying force on the first seal member 731 and the second seal member 735 in opposite directions. The first storage volume 706a can be open to, merged or combined with the second storage volume 706b to enable the specimen S to move between the volumes and/or enable other fluids or materials stored within the second storage volume 706b to mix with the specimen S. For example, at least one of the first storage volume 706a or the second storage volume 706b can be coupled to a port, as described above, to introduce fluids or other materials into one or both of the storage volumes 706a, 706b. In some embodiments, the second storage volume 706b can be prefilled with preservation fluid such that after tissue specimen S is inserted into the first storage volume 706a, the first seal member 731 and the second seal member 735 can be separated to introduce the preservation fluid from the second storage volume 706b into the first storage volume 706a. By providing the preservation fluid in the second storage volume 706b, separate steps for connecting and introducing preservation fluid into the container is eliminated, thereby removing a step for potential user error. The second storage volume 706b with prefilled preservation fluid can also minimize the time between placement of a specimen in the container and introduction of the preservation fluid with the specimen, thereby improving preservation of the harvested tissue. The prefilled volume of preservation fluid can enable the flexible container 700 to be used in remote or off-site locations, and can minimize introduction of foreign matter into the flexible container 700 by eliminating separate steps of connecting and filling the container with an external source of preservation fluid after the specimen has been placed in the container. The flexible container 700 can be used, for example, in the field for prompt on-site harvesting of tissues from a donor, and the on-site harvesting can take place outside of a hospital or laboratory setting.

When the flexible container 700 is in a fourth (or retrieving) configuration, as shown in FIG. 14D, the second self-sealing mechanism 730 can be opened to separate the first layer L₁ from the second layer L₂ at the second end portion to form an exit opening 708 into the first and second storage volumes 706a, 706b. In this fourth configuration, the tissue specimen S can be removed from the flexible container 700 via the exit opening 708. Additionally, or alternatively, the flexible container 700 can include a peelable seal and/or perforated tear opening at the second end portion 702 to form the exit opening 708.

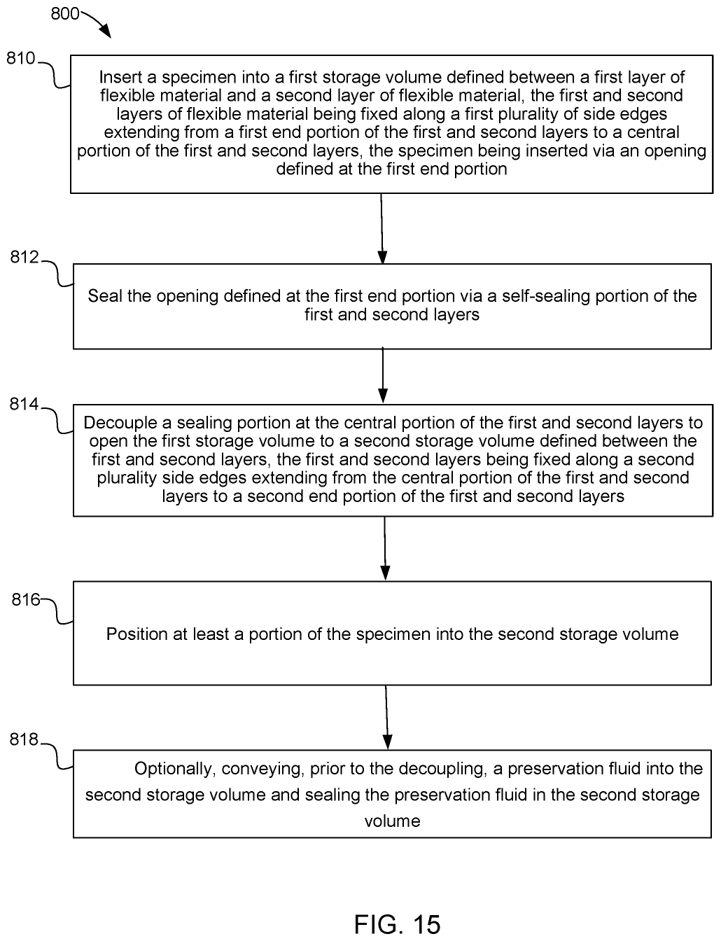

Any of the flexible containers described herein can be used to perform any suitable methods of tissue storage. In some embodiments, any of the containers described herein can be used to seal a tissue specimen at the point of harvest without the use of external sealing equipment. For example, FIG. 15 is a flow chart showing a method 800 of storing tissue specimen according to an embodiment. The method 800 includes inserting 810 a specimen into a first storage volume of a container defined between a first layer of flexible material and a second layer of flexible material. The container can be, for example, the container 700 described above and the first and second layers of flexible material are fixed along a first plurality of side edges extending from a first end portion of the first and second layers to a central portion of the first and second layers. The specimen is inserted via an entry opening defined at the first end portion. The method 800 includes sealing 812 the opening defined at the first end portion via a self-sealing portion of the first and second layers. The method 800 includes decoupling 814 a sealing portion at the central portion of the first and second layers to open the first storage volume to a second storage volume defined between the first and second layers, the first and second layers being fixed along a second plurality side edges extending from the central portion of the first and second layers to a second end portion of the first and second layers. In some embodiments, the method 800 further includes positioning 816 a portion of the specimen into the second storage volume. In some embodiments, the method 800 further includes optionally conveying 818, prior to the decoupling 814, a preservation fluid into the second storage volume and sealing the preservation fluid in the second storage volume.

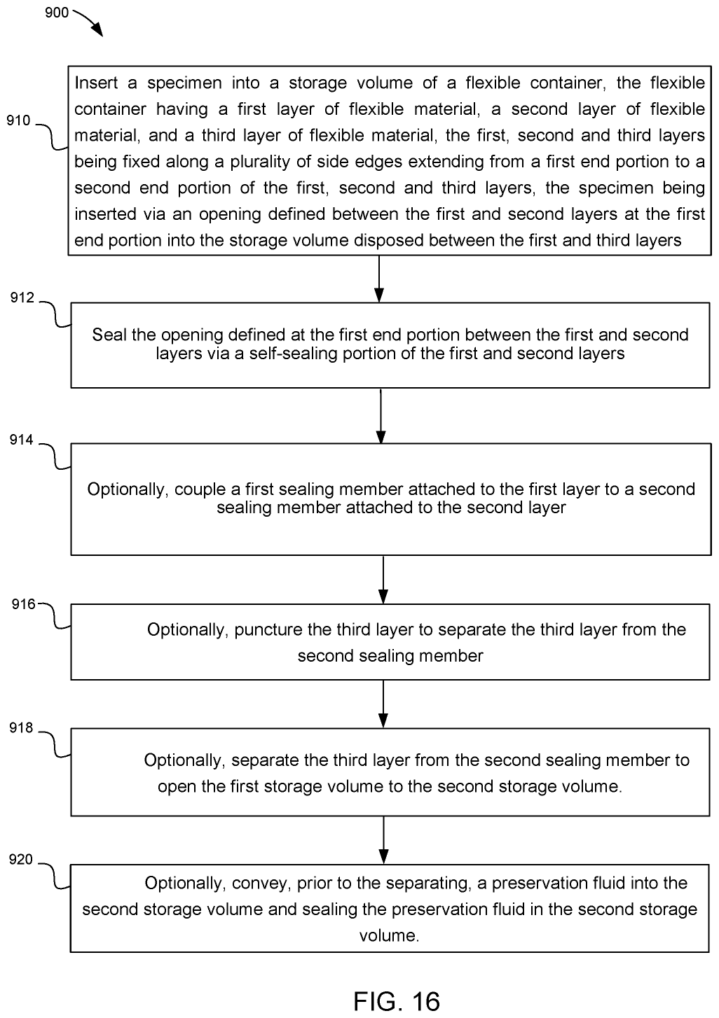

FIG. 16 is a flow chart showing a method 900 of storing tissue specimen according to an embodiment. The method 900 includes inserting 910 a specimen into a storage volume of a flexible container. The container can be, for example, the container 600 described above having a first layer of flexible material, a second layer of flexible material, and a third layer of flexible material, the first, second and third layers being fixed along a plurality of side edges extending from a first end portion to a second end portion of the first, second and third layers. The specimen is inserted via an opening defined between the first and second layers at the first end portion into the storage volume disposed between the first and third layers. The method 900 includes sealing 912 the opening defined at the first end portion between the first and second layers via a self-sealing portion of the first and second layers. In some embodiments, the sealing optionally includes coupling 914 a first sealing member attached to the first layer to a second sealing member attached to the second layer. In some embodiments, the container 600 includes a second storage volume disposed between the second and third layers, and the second sealing member is attached to the second and third layers at the first end portion. In such embodiments, the coupling 914 opti includes puncturing 916 the third layer to separate the third layer from the second sealing member. In some embodiments, the separating includes opening 918 the first storage volume to the second storage volume. In some embodiments, the method 900 further includes conveying 920, prior to the separating, a preservation fluid into the second storage volume and sealing the preservation fluid in the second storage volume.

While various embodiments have been described above, it should be understood that they have been presented by way of example only, and not limitation. Where methods and/or schematics described above indicate certain events and/or flow patterns occurring in certain order, the ordering of certain events and/or operations may be modified. While the embodiments have been particularly shown and described, it will be understood that various changes in form and details may be made.

Although various embodiments have been described as having particular features and/or combinations of components, other embodiments are possible having a combination of any features and/or components from any of embodiments as discussed above. Aspects have been described in the general context of medical devices, and more specifically tissue packaging devices, but inventive aspects are not necessarily limited to use in medical devices and tissue packaging.

1. An apparatus comprising:
a first layer of flexible material; and
a second layer of flexible material, the second layer being coupled to the first layer to form a container defining a storage volume bounded by a first end portion of the container, a second end portion of the container, a first side edge of the container, and a second side edge of the container;
wherein:
the first layer and the second layer defining an opening along a segment extending along the first end portion and the first side edge;
the first layer and the second layer together include a self-sealing portion along the segment extending along the first end portion and the first side edge.

2. The apparatus of claim 1, wherein the segment extending along the first end portion and the first side edge includes a curved segment section.

3-4. (canceled)

5. The apparatus of claim 1, wherein:
the first layer of flexible material defines a first outer perimeter;
the second layer of flexible material defines a second outer perimeter; and
the first outer perimeter is different from the second outer perimeter.

6. The apparatus of claim 5, wherein:
a portion of first outer perimeter that extends beyond the second outer perimeter is a first handle portion; and
a portion of the second outer perimeter that extends beyond the first outer perimeter is a second handle portion.

7-13. (canceled)

14. The apparatus of claim 1, wherein the self-sealing portion includes a plurality of sealing mechanisms.

15. (canceled)

16. The apparatus of claim 14, wherein:
at least one of the plurality of sealing mechanisms is configured to be a single-use sealing mechanism; and
the self-sealing portion includes an indicator that identifies when the plurality of sealing mechanisms are in a sealed configuration, or identifies when the plurality of sealing mechanisms have been opened after an initial instance of being fully sealed.

17. (canceled)

18. An apparatus comprising:
a first layer of flexible material; and
a second layer of flexible material, the second layer being coupled to the first layer to form a container defining a storage volume bounded by a first end portion of the container, a second end portion of the container, and a plurality of side edges of the container, the first layer and the second layer defining an opening at the first end portion of the container; and
a self-sealing portion having a plurality of sealing mechanisms, each of the plurality of sealing mechanisms being configured to close and seal the opening at the first end portion.

19. The apparatus of claim 18, wherein the self-sealing portion comprises a set of complimentary hook and grooves and a set of complimentary U-shaped ribs.

20-26. (canceled)

27. The apparatus of claim 18, wherein:
at least one of the plurality of sealing mechanisms is configured to be a single-use sealing mechanism; and
the self-sealing portion includes an indicator that identifies when the plurality of sealing mechanisms are in a sealed configuration, or identifies when the plurality of sealing mechanisms have been opened after an initial instance of being fully sealed.

28. (canceled)

29. The apparatus of claim 18, wherein the first layer and the second layer are configured to be coupled at a central portion of the container to separate the storage volume into a first partition and a second partition, the central portion being located between the first end portion and the second end portion.

30. The apparatus of claim 29, wherein the self-sealing portion at the first end portion is a first self-sealing portion, the apparatus further comprising:
a second self-sealing portion at the central portion of the container, the second self-sealing portion including a first seal member and a second seal member, the first seal member configured to matingly engage the second seal member to fluidically isolate the first partition from the second partition.

31-37. (canceled)

38. The apparatus of claim 30, wherein:
the first seal member includes a base portion, a neck portion and a head portion, the neck portion being disposed between the base portion and the head portion;
the second seal member includes a base portion, a first shoulder, a second shoulder, and a groove defined between the first shoulder and the second shoulder; and
the groove of the second seal member is configured to receive and retain the head portion of the first seal member, the first layer at the central portion of the container, and the second layer at the central portion of the container.

39. (canceled)

40. An apparatus comprising:
a first layer of flexible material;
a second layer of flexible material, the second layer of flexible material being coupled to the first layer to form a container bounded by a first end portion of the container, a second end portion of the container and a plurality of side edges of the container, the first layer and the second layer defining an opening at the first end portion; and
a third layer of flexible material, the third layer of flexible material being attached to and configured to selectively decouple from at least one of the first layer or the second layer, the third layer of flexible material being disposed between the first layer and the second layer to define a first storage volume of the container between the first layer and the third layer and to define a second storage volume of the container between the second layer and the third layer; and
a self-sealing portion having a plurality of sealing mechanisms to close and seal the opening at the first end portion.

41. The apparatus of claim 40, wherein:
the self-sealing portion includes a first sealing member secured to the first layer; and
the self-sealing portion includes a second sealing member secured to the second layer, the first sealing member and the second sealing member being configured to interlock together to seal the opening at the first end portion.

42. The apparatus of claim 41, wherein:
the first sealing member includes a first hook and groove channel;
the second sealing member includes a first corresponding hook and groove channel configured to interlock with the first hook and groove channel of the first sealing member;
the first sealing member includes a second hook and groove channel; and
the second sealing member includes a second corresponding hook and groove channel configured to interlock with the second hook and groove channel of the first sealing member.

43-46. (canceled)

47. The apparatus of claim 40, wherein:
the opening is an entry opening;
the self-sealing portion is a first self-sealing portion; and
the first and second layers define an exit opening at the second end portion of the container; and
the apparatus further comprises a second self-sealing portion configured to seal the exit opening.

48. The apparatus of claim 47, wherein:
the second self-sealing portion includes a first sealing member attached to the first layer at the second end portion; and
a second sealing member attached to the second layer at the second end portion.

49. (canceled)

50. The apparatus of claim 48, wherein:
the third layer includes a third sealing member at the second end portion; and
the third sealing member is configured to nest between the first and second sealing member when the first and second sealing member are interlocked.

51-56. (canceled)

57. The apparatus of claim 40, further comprising a first flap portion attached to the first layer at the first end portion and a second flap portion attached to the third layer at the first end portion, the first and second flap portions extending beyond the opening at the first end portion.

58-59. (canceled)

60. The apparatus of claim 40, wherein:
the first layer includes one or more of fluorinated ethylene propylene (FEP), Nylon, polytetrafluoroethylene (PTFE), or polyethylene terephthalate with the addition of glycol (PETG); and
the second layer includes one or more of fluorinated ethylene propylene (FEP), Nylon, polytetrafluoroethylene (PTFE), or polyethylene terephthalate with the addition of glycol (PETG).

61-67. (canceled)
